(19) United States
(12) Patent Application Publication
(10) Pub. No.: US 2008/0261926 A1
(43) Pub. Date: Oct. 23, 2008

(54) Pharmaceutical Calcimimetics

(76) Inventors: Julie F. Liu, Lexington, MA (US); Jillian Imagire, Lexington, MA (US)

Correspondence Address: FISH & RICHARDSON P.C., PO BOX 1022, MINNEAPOLIS, MN, 55440-1022, US

(21) Appl. No.: 12/060,371

(22) Filed: Apr. 1, 2008

Related U.S. Application Data
(60) Provisional application No. 60/909,565, filed on Apr. 2, 2007.

(51) Int. Cl. A61K 31/135; A61K 31/59; A61P 13/12; A61P 9/00; A61P 19/00

(52) U.S. Cl. 514/167; 514/650

(57) ABSTRACT

This invention relates to novel calcimimetic compounds, their derivatives, pharmaceutically acceptable salts, solvates, and hydrates thereof. This invention also provides compositions comprising a compound of this invention and the use of such compositions in methods of treating diseases and conditions that are beneficially treated by binding to, and modulating the sensitivity of, calcium receptors on the parathyroid gland.

### CROSS-REFERENCE TO RELATED APPLICATION

This application claims priority under 35 U.S.C. § 119 to U.S. Provisional Application Ser. No. 60/909,565, filed Apr. 2, 2007, the entire contents of which are incorporated by reference herein.

### BACKGROUND

This invention relates to novel calcimimetic compounds, their derivatives, pharmaceutically acceptable salts, solvates, and hydrates thereof. This invention also provides compositions comprising a compound of this invention and the use of such compositions in methods of treating diseases and conditions that are beneficially treated by administration of compounds that bind to, and/or modulate the sensitivity of, calcium receptors on the parathyroid gland.

Cinacalcet is known by the chemical name 3-(3-(trifluoromethyl)phenyl)-N—((R)-1-(naphthalen-5-yl)ethyl)propan-1-amine. The hydrochloride salt of cinacalcet is known as Sensipar™ and by the chemical name N-[1(R)-(1-naphthyl)ethyl]-N-[3-[3-(trifluoromethyl)phenyl]propyl]amine hydrochloride. Cinacalcet and its salts each have one stereogenic center, in the R configuration, at the naphthalenic, methine carbon.

Cinacalcet is an agent that decreases the levels of parathyroid hormone (PTH), calcium, and phosphorous in the blood by binding to the transmembrane region of the calcium-sensing receptors (CaR) on the surface of the parathyroid gland. This binding causes these receptors to adopt a different structural configuration, which is more sensitive to extracellular calcium (e.g., calcium ions in the blood). This increase in sensitivity causes greater stimulation of these receptors for a given concentration of extracellular calcium ions. The increased stimulation of CaR causes a decrease in the amount of parathyroid hormone (PTH) that is secreted by the parathyroid gland because stimulation of CaR inhibits the secretion of PTH, which leads to a decrease in the concentration of extracellular calcium.

Cinacalcet is indicated for the treatment of secondary hyperparathyroidism and hypercalcemia. Cinacalcet is approved for use in both (a) reducing elevated levels of PTH in people with chronic renal disease who are on dialysis; and (b) lowering calcium levels in patients with cancer of the parathyroid gland.

Cinacalcet is in the clinical trial phase for the treatment of the following diseases and conditions: primary hyperparathyroidism (e.g., familial hyperparathyroidism); secondary hyperparathyroidism; kidney disease (e.g., chronic kidney disease (CKD)); hypophosphatemic rickets; anemia; hypercalcemia; end stage renal disease; calcification (e.g., coronary artery calcification and vascular calcification); cardiovascular disease; nephrology; Paget's disease; osteoporosis; hypertension; and renal osteodystrophy.

Despite the beneficial activities of cinacalcet, there is a continuing need for new compounds to treat the aforementioned diseases and conditions.

### BRIEF DESCRIPTION OF THE DRAWINGS

FIG. 1 depicts the stability of D-cinacalcet (Compound 100, Example 7) as compared to Cinacalcet in Human Liver Microsomes (HLMs).

### DETAILED DESCRIPTION

### Definitions

The terms “ameliorate” and “treat” are used interchangeably and include therapeutic and/or prophylactic treatment. Both terms mean decrease, suppress, attenuate, diminish, arrest, or stabilize the development or progression of a disease (e.g., a disease or disorder delineated herein).

The term “disease” means any condition or disorder that damages or interferes with the normal function of a cell, tissue, or organ.

It is well known that some variation of natural isotopic abundance occurs in a synthesized compound depending upon the origin of chemical materials used in the synthesis. Thus, a preparation of cinacalcet will inherently contain small amounts of deuterated isotopologues. The concentration of such naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Ganes L Z et al., Comp Biochem Physiol Mol Integr Physiol 1998, 119:725. In a compound of this invention, when a particular position is designated as having deuterium, it is understood that the abundance of deuterium at that position is enriched beyond the natural abundance of deuterium, which is 0.015%. A position designated as having deuterium typically has a minimum isotopic enrichment factor of at least 3000 (45% deuterium incorporation).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention, any atom not specifically designated as a particular isotope is meant to represent any stable isotope of that atom unless otherwise stated. Unless otherwise stated, when a position is designated specifically as “H” or “hydrogen,” the position is understood to have a hydrogen atom at its natural isotopic abundance (i.e., 99.985%).

The term “isotopologue” refers to a species that has the same chemical structure and formula as a specific compound of this invention, with the exception of the isotopic composition at one or more positions, e.g., H vs. D. Thus an isotopologue differs from a specific compound of this invention in the isotopic composition thereof.

The term “compound,” as used herein, is also intended to include any salts (e.g., hydrochloride salts), solvates, or hydrates thereof.

A salt of a compound of this invention is formed between an acid (e.g., hydrochloric acid) and a basic group of the compound (e.g., an amino moiety) or a base and an acidic group of the compound, such as a carboxylic acid moiety. Accordingly, in one embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not normally toxic at concentrations that result from administration of the salt to a recipient (e.g., a subject).

Acids commonly employed to form pharmaceutically acceptable salts include inorganic acids, such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids, such as hydrochloric acid and hydrobromic acid, and especially those formed with organic acids, such as maleic acid.

As used herein, the term “tosyl” or its abbreviation “Ts” means para-toluene sulfonyl (e.g., TsCl means para-toluene sulfonyl chloride).

As used herein, reference to a ruthenium catalyst by the reference number “CAS [201816-42-4]” means the ruthenium catalyst of the following structure:

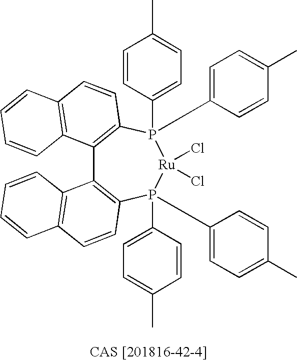

The term “leaving group” as used herein means any group susceptible to a substitution reaction. See, Jones M Jr., Organic Chemistry, W. W. Norton & Co. (1997) 259. Conversion of a moiety in a compound to an “appropriate leaving group” means converting said moiety to a moiety that is more susceptible to substitution.

As used herein, the term “hydrate” means a compound which further includes a stoichiometric or non-stoichiometric amount of water bound non-covalently.

As used herein, the term “solvate” means a compound which further includes a stoichiometric or non-stoichiometric amount of any solvent (e.g., water, acetone, ethanol, methanol, 2-propanol, dichloromethane, ethers, amines, etc.), bound non-covalently.

The compounds of this invention (e.g., compounds of Formula I), contain at least one stereogenic center. Additionally, depending on the deuterium composition of these compounds, the compounds of this invention may have additional stereogenic centers. Accordingly, a compound of this invention may exist as either a stereoisomerically pure compound (e.g., one of (S) or (R)), or as a mixture of two or more stereoisomers. A compound of the present invention will include both mixtures of stereoisomers (e.g., racemic mixtures) and also individual respective stereoisomers that are substantially free from another possible stereoisomer. The term “substantially free of other stereoisomers” as used herein means less than 25% of other stereoisomers, less than 10% of other stereoisomers, less than 5% of other stereoisomers, less than 2% of other stereoisomers, or less than “X”% of other stereoisomers (wherein X is a number between 0 and 100, inclusive) are present. Methods of obtaining or synthesizing an individual stereoisomer for a given compound are well known in the art and may be applied as practicable to final compounds or to starting material or intermediates.

The term “stable compounds,” as used herein, refers to compounds that possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to a deuterium atom.

The term “stereoisomer” refers to a molecule capable of existing in more than one spatial atomic arrangement for a given atomic connectivity (e.g., enantiomers, meso compounds, and diastereomers). As used herein, the term “stereoisomer” means either or both enantiomers and diastereomers.

Throughout this specification, reference to “each R” includes, independently, any “R” group (e.g., R¹ and R²) where applicable.

### Therapeutic Compounds

The present invention provides an isolated compound of Formula I:

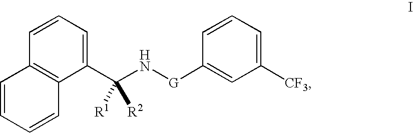

, or a salt, hydrate, or solvate thereof:
wherein,

R¹ is selected from CH₃, CH₂D, CHD₂, and CD₃;

R² is selected from H and D;

G is †-(CHₘD₂₋ₘ)(CHₙD₂₋ₙ)(CHₚD₂₋ₚ)-, wherein m, n, and p are each independently selected from 0, 1, and 2, and the † represents the portion of G attached to the NH moiety in the compound; and

the compound comprises at least one deuterium atom at R¹, R², or G.

In one embodiment, R¹ is CD₃ or CH₃.

In another embodiment, R¹ is CD₃.

In another embodiment of the invention, R¹ is CH₃

In another embodiment, R² is D.

In another embodiment, R¹ is CD₃ and R² is D.

In another embodiment, each of m, n, and p is independently selected from 0 or 2.

In another embodiment, each of m, n, and p is 2.

In another embodiment, n is 0 and each of m and p is 2.

In another embodiment, n and p are both 0 and m is 2.

In another embodiment, R¹ is CD₃, m is 2, and each of n and p is independently selected from 0 or 2.

In another embodiment, R¹ is CD₃, R² is D, m is 2, and each of n and p is independently selected from 0 or 2.

In another embodiment, R¹ is CH₃, m is 2, and each of n and p is independently selected from 0 or 2.

In yet another embodiment, the compound of Formula I is selected from any one of the compounds set forth in Table 1, wherein the † represents the portion of G attached to the NH moiety in the compound:

TABLE 1
Exemplary Embodiments of Formula I

| Compound | R¹ | R² | G |
|---|---|---|---|
| 100 | CD₃ | D | †CD₂CD₂CD₂ |
| 101 | CD₃ | D | †CD₂CH₂CD₂ |
| 102 | CD₃ | D | †CD₂CH₂CH₂ |
| 103 | CH₃ | D | †CD₂CD₂CD₂ |
| 104 | CH₃ | D | †CD₂CH₂CD₂ |
| 105 | CH₃ | D | †CD₂CH₂CH₂ |
| 106 | CH₃ | H | †CD₂CH₂CH₂ |

In another set of embodiments, any atom not designated as deuterium in any of the embodiments set forth above is present at its natural isotopic abundance.

In another set of embodiments, the compound of Formula I is isolated or purified, e.g., the compound of Formula I is present at a purity of at least 50% by weight (e.g., at least 55%, 60%, 65%, 70%, 75%, 80%, 85%, 90%, 95%, 97%, 98%, 98.5%, 99%, 99.5% or 99.9%) of the total amount of isotopologues of Formula I present. Thus, in some embodiments, a composition comprising a compound of Formula I can include a distribution of isotopologues of the compound, provided at least 50% of the isotopologues by weight are the recited compound.

In some embodiments, any position in the compound of Formula I designated as having D has a minimum deuterium incorporation of at least 45% (e.g., at least 52.5%, at least 60%, at least 67.5%, at least 75%, at least 82.5%, at least 90%, at least 95%, at least 97%, at least 99%, or at least 99.5%) at the designated position(s) of the compound of Formula I. Thus, in some embodiments, a composition comprising a compound of Formula I can include a distribution of isotopologues of the compound, provided at least 45% of the isotopologues include a D at the designated position(s).

In some embodiments, a compound of Formula I is “substantially free of” other isotopologues of the compound, e.g., less than 50%, less than 25%, less than 10%, less than 5%, less than 2%, less than 1%, or less than 0.5% of other isotopologues are present.

The synthesis of compounds of Formula I can be readily achieved by synthetic chemists of ordinary skill. Relevant procedures and intermediates are disclosed, for instance, in U.S. Pat. Nos. 6,313,146; 6,211,244; 6,031,003; and 6,011,068; U.S. Patent Application No. 2007/0043243; as well as PCT Publication Nos.: WO 06/127932; WO 06/127941; WO 06/127933; and WO 06/125026

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Methods of Synthesis

A method for synthesizing compounds of Formula I is depicted in Scheme 1.

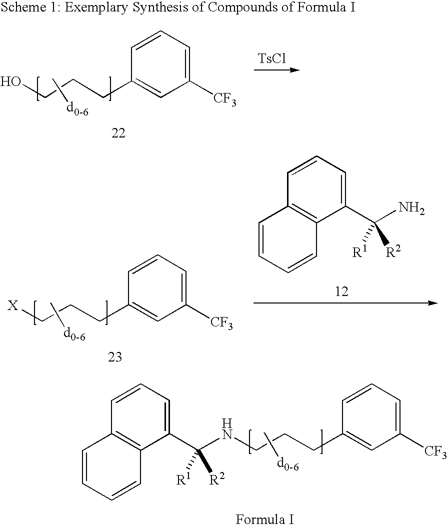

According to Scheme 1, the synthesis of compounds of Formula I commences from alcohol 22. As a preliminary matter, compound 22 represents several deuterated compound variants including

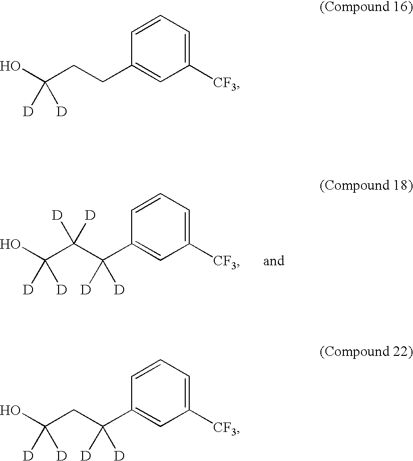

that can be used to prepare a number of compounds of Formula I. The alcohol moiety in compound 22 is converted to a leaving group such as a tosylate using tosyl chloride in the presence of an amine base such as triethyl amine or the like. The leaving group in the resulting compound 23 is then displaced with chiral amine 12 (R¹═CH₃, CH₂D, CHD₂, or CD₃) to afford a compound of formula I.

The preparation of chiral amine 12 and compound 22 are depicted in the following schemes.

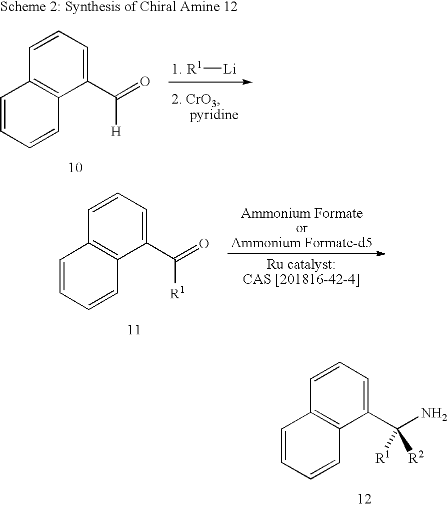

As depicted in Scheme 2, addition of a selected alkyl lithium reagent R¹Li (R¹ is CH₃, CH₂D, CHD₂, or CD₃), to 1-naphthaldehyde 10, followed by oxidation with an oxidizing agent such as CrO₃, yields the corresponding ketone 11. Reductive amination of 11 with a ruthenium catalyst, such as CAS [201816-42-4], stereoselectively provides the chiral amine 12. See Kadyrov R et al, Ang Chem Int Ed 2003, 42(44): 5472.

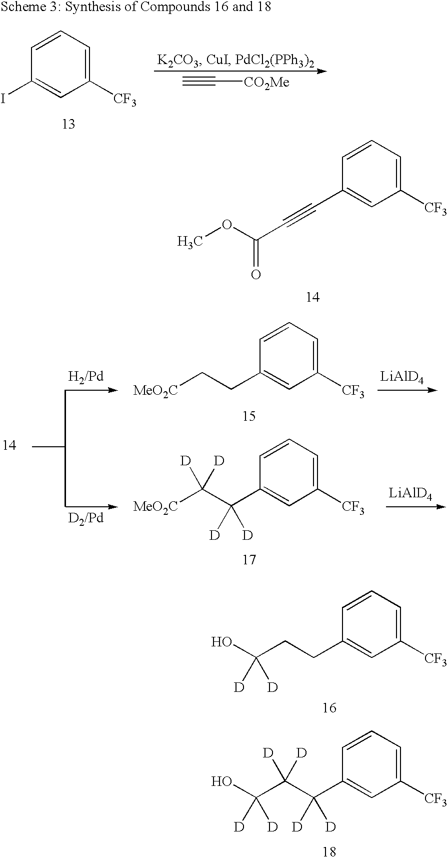

As depicted in Scheme 3, the synthesis of the requisite alcohols 16 and 18 commences with the preparation of 3-(3-(trifluoromethyl)phenyl)propiolate 14 by coupling of commercially available 1-(trifluoromethyl)-3-iodobenzene 13 with methyl 2-propynoate following the method of Eckert. Synth. Comm. 1998, 28(2): 327-335. Propiolate 14 can be converted to the corresponding optionally-deuterated aliphatic ester (compounds 15 or 17) by palladium-catalyzed hydrogenation or deuteration (that is, hydrogenation using deuterium gas). Hattori K, Tetrahedron 2001, 57(23): 4817-4824). Thus, palladium-catalyzed hydrogenation of compound 14 with H₂ affords compound 15, and palladium-catalyzed deuteration of compound 14 with D₂ affords compound 17. The aliphatic esters 15 and 17 can be reduced with LiAlD₄ to provide the corresponding primary alcohols 16 and 18. The reductions of 15 or 17 may be carried out with LiAlH₄ in place of LiAlD₄. In such a case, the carbon bearing the primary alcohol in the reduction product comprises H instead of D (i.e., CH₂ instead of CD₂).

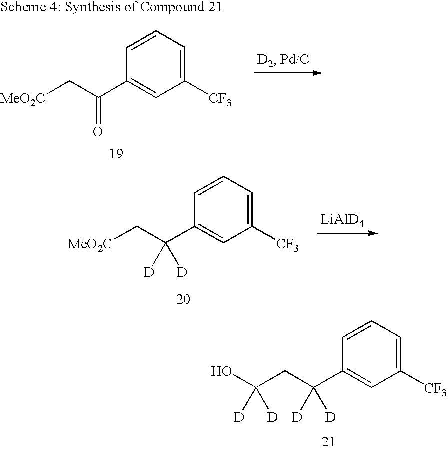

As depicted in Scheme 4, compound 19 may be reduced with D₂ in the presence of palladium on carbon (Pd/C) to afford the deuterated ester 20. Reduction of ester 20 using, for example, LiAlD₄ affords deuterated aliphatic alcohol 21.

Scheme 5 depicts an alternative exemplary synthesis of compounds of Formula I.

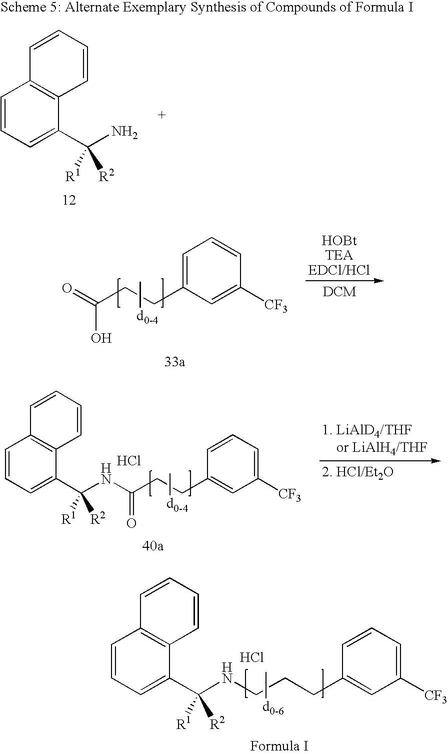

According to Scheme 5, chrial amine 12 is coupled with a carboxylic acid of the general formula 33a using 1-hydroxybenzotriazole/EDCI to afford the optionally deuterated amide 40a. Reduction of amide 40a with LiAlD₄ or LiAlH₄, followed by treatment with HCl, provides appropriately deuterated compounds of Formula I.

The specific approaches and compounds shown above are not intended to be limiting. The chemical structures in the schemes herein depict variables that are hereby defined commensurately with chemical group definitions (moieties, atoms, etc.) of the corresponding position in the compounds of Formula I herein, whether identified by the same variable name (e.g., R₁, R₂, etc.) or not. The suitability of a chemical group in a compound structure for use in the synthesis of another compound is within the knowledge of one of ordinary skill in the art. Additional methods of synthesizing compounds of Formula I and their synthetic precursors, including those within routes not explicitly shown in schemes herein, are within the means of chemists of ordinary skill in the art. Methods for optimizing reaction conditions and, if necessary, minimizing competing by-products, are known in the art. Reaction optimization and scale-up may advantageously utilize high-speed parallel synthesis equipment and computer-controlled microreactors (e.g., Design And Optimization in Organic Synthesis, 2^{nd} Edition, Carlson R, Ed, Elsevier Science Ltd. (2005); Jähnisch, K et al, Angew. Chem. Int. Ed. Engl. 2004, 43: 406; and references therein). In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google or keyword databases such as the U.S. Patent and Trademark Office text database.

The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, Ed., Encyclopedia of Reagents for Organic Synthesis, John Wiley and Sons (1995) and subsequent editions thereof.

Combinations of substituents and variables envisioned by this invention are only those that result in the formation of stable compounds.

### Pharmaceutical Compositions

The invention also provides pyrogen-free compositions comprising an effective amount of a compound of Formula I (e.g., including any of the formulae herein), including pharmaceutically acceptable salts, solvates, or hydrates; and an acceptable carrier. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

In some embodiments, a pharmaceutical composition includes an effective amount of a compound of Formula I and an acceptable carrier, wherein any position in the compound of Formula I designated as having D has a minimum deuterium incorporation of at least 45% (e.g., at least 52.5%, at least 60%, at least 67.5%, at least 75%, at least 82.5%, at least 90%, at least 95%, at least 97%, at least 99%, or at least 99.5%) in the composition.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, e.g., Remington's Pharmaceutical Sciences, Mack Publishing Company, Philadelphia, Pa., 17th Ed. (1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g., U.S. Pat. No. 6,803,031.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers, or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids, or combinations thereof to impart controlled release characteristics in the composition. Coatings for invasive devices are to be included within the definition of pharmaceutically acceptable carrier, adjuvant, or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules, and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible (e.g., because of removal from the subject or surgical procedure) such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as cinacalcet (e.g., modulation of the activity of calcium receptors on the parathyroid gland).

In one embodiment, the second therapeutic agent is an agent useful in the treatment or prevention of one or more of the following diseases or conditions: primary hyperparathyroidism (e.g., familial hyperparathyroidism); secondary hyperparathyroidism; kidney disease (e.g., chronic kidney disease); hypophosphatemic rickets; anemia; hypercalcemia; end stage renal disease; calcification (e.g., coronary artery calcification and vascular calcification); cardiovascular disease; nephrology; Paget's disease; osteoporosis; hypertension; and renal osteodystrophy.

In another embodiment, the second therapeutic agent is selected from vitamin D, vitamin D analogues, phosphate binders (e.g., aluminum, calcium, or lanthanum salts, and calcium-containing phosphate binders, such as Aluminum hydroxide (Alucaps®); Calcium carbonate (Calcichew®, Titralac®); Calcium acetate (Phosex®, PhosLo®); Lanthanum carbonate (Fosrenol®) Sevelamer (Renagel®, Renvela®)), and agents used to raise serum calcium concentrations.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., Cancer Chemother. Rep. 1966, 50: 219. Body surface area may be approximately determined from height and weight of the subject. See, e.g., Scientific Tables, Geigy Pharmaceuticals, Ardsley, N.Y., (1970) 537.

In one embodiment, an effective amount of a compound of this invention can range from about 0.01 mg/kg to about 50 mg/kg.

In another embodiment, an effective amount of a compound of this invention can range from about 0.04 mg/kg to about 26 mg/kg.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for cinacalcet.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., Eds., Pharmacotherapy Handbook, 2nd Ed., Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of calcium receptors (e.g., increasing the sensitivity of calcium receptors on the parathyroid glands), comprising contacting a calcium receptor (e.g., a calcium receptor of a mammal, such as a human, horse, cow, pig, sheep, dog, cat, mouse, rat, or monkey) with one or more compounds of Formula I herein.

In another embodiment, the invention provides a method of treating a subject suffering from, or susceptible to, a disease that is beneficially treated by cinacalcet, comprising the step of administering to said subject an effective amount of a compound or a composition of this invention. Such diseases are well known in the art and are disclosed in, but not limited to, the following patents and published applications: U.S. Pat. Nos. 6,011,068; 6,031,003; 6,211,244; and 6,313,146; U.S. Patent Application No. 2005/147669; and PCT Publication Nos. WO 06/125026; WO 05/034928; WO 06/127932; WO 06/127941; and WO 06/127933.

In another embodiment, the method of this invention is used to treat a subject suffering from or susceptible to one or more of the following diseases and conditions: primary hyperparathyroidism (e.g., familial hyperparathyroidism), secondary hyperparathyroidism; kidney disease (e.g., chronic kidney disease), hypophosphatemic rickets, anemia, hypercalcemia, end stage renal disease, calcification (e.g., coronary artery calcification and vascular calcification), cardiovascular disease, nephrology, Paget's disease, osteoporosis, hypertension, and renal osteodystrophy. In particular embodiments, the method of this invention is used to treat a subject suffering from or susceptible to a disease or condition selected from secondary hyperparathyroidism and hypercalcemia.

Identifying a subject in need of treatment with a compound of this invention according to the methods of this invention can be in the judgment of a subject or a health care professional and can be subjective (e.g., opinion) or objective (e.g., measurable by a test or diagnostic method).

In another embodiment, any of the above methods of treatment comprises the further step of co-administering to said subject one or more second therapeutic agents. The choice of second therapeutic agent may be made from any second therapeutic agent known to be useful for co-administration with cinacalcet. Examples of conditions and diseases that may be treated with a compound of this invention (e.g., compounds of Formula I) in combination with a second therapeutic agents include, but are not limited to primary hyperparathyroidism (e.g., familial hyperparathyroidism), secondary hyperparathyroidism, kidney disease (e.g., chronic kidney disease), hypophosphatemic rickets, anemia, hypercalcemia, end stage renal disease, calcification (e.g., coronary artery calcification and vascular calcification), cardiovascular disease, nephrology, Paget's disease, osteoporosis, hypertension, and renal osteodystrophy. More specific examples of second therapeutic agents that may be employed in the methods of this invention are those set forth above for use in combination compositions.

In one embodiment, the combination therapies of this invention include treatment of the following diseases and conditions by administering a compound of Formula I: primary hyperparathyroidism (e.g., familial hyperparathyroidism), secondary hyperparathyroidism, kidney disease (e.g., chronic kidney disease), hypophosphatemic rickets, anemia, hypercalcemia, end stage renal disease, calcification (e.g., coronary artery calcification and vascular calcification), cardiovascular disease, nephrology, Paget's disease, osteoporosis, hypertension, and renal osteodystrophy.

In another embodiment, the combination therapies of this invention include treatment of secondary hyperparathyroidism and/or hypercalcemia by administering a compound of Formula I.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., Eds., Pharmacotherapy Handbook, 2nd Ed., Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be in cases in which the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In another embodiment, this invention provides for the use of a compound of Formula I, alone or together with one of the above-described second therapeutic agents, in the manufacture of a medicament, either in a single composition or in separate dosage forms, for treating a disease that is beneficially treated by cinacalcet. Such diseases are well known in the art and include, but are not limited to primary hyperparathyroidism (e.g., familial hyperparathyroidism), secondary hyperparathyroidism, kidney disease (e.g., chronic kidney disease), hypophosphatemic rickets, anemia, hypercalcemia, end stage renal disease, calcification (e.g., coronary artery calcification and vascular calcification), cardiovascular disease, nephrology, Paget's disease, osteoporosis, hypertension, and renal osteodystrophy.

### Diagnostic Methods and Kits

As indicated, the compounds of this invention (i.e., the compounds of Formula I), and compositions comprising them, are useful for treating or lessening the severity of disorders that are effectively treated by binding to, and modulating the sensitivity of, calcium receptors on the parathyroid gland. The compounds and compositions of this invention are also useful as reagents in methods for determining the concentration of cinacalcet in solution, examining the metabolism of cinacalcet, studying the relationship between the concentration of an ion (e.g., calcium ion) and the concentration of PTH in a mammal, studying the relationship between the administration of cinacalcet and the concentration of an ion (e.g., calcium ion) in a mammal, studying the relationship between administration of cinacalcet and the concentration of PTH in a mammal, and other analytical studies. Additional utility of compounds of Formula I include their use as internal standards to determine the true concentration(s) of marker compounds (e.g., Cinacalcet) in biological matrices, such as plasma.

According to one embodiment, the invention provides a method of determining the concentration, in a solution or a biological sample, of cinacalcet, comprising the steps of:

a) adding a known concentration of a compound of Formula I to the solution or biological sample;

b) subjecting the solution or biological sample to a measuring device that distinguishes cinacalcet from a compound of Formula I;

c) calibrating the measuring device to correlate the detected quantity of the compound of Formula I with the known concentration of the compound of Formula I added to the biological sample or solution; and

d) measuring the quantity of cinacalcet in the biological sample with said calibrated measuring device; and

e) determining the concentration of cinacalcet in the solution of sample using the correlation between detected quantity and concentration obtained for a compound of Formula I.

Measuring devices that can distinguish cinacalcet from the corresponding compound of Formula I include any measuring device that can distinguish between two compounds that differ from one another in isotopic abundance. Exemplary measuring devices include a mass spectrometer, NMR spectrometer, or IR spectrometer.

In another embodiment, the invention provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the invention provides a method of evaluating the metabolic stability of a compound of Formula I in a subject following administration of the compound of Formula I. This method comprises the steps of obtaining a serum, urine, or feces sample from the subject at a period of time following the administration of the compound of Formula I to the subject; and comparing the amount of the compound of Formula I with the metabolic products of the compound of Formula I in the serum, urine, or feces sample.

The present invention also provides kits for use to treat primary hyperparathyroidism (e.g., familial hyperparathyroidism), secondary hyperparathyroidism, kidney disease (e.g., chronic kidney disease), hypophosphatemic rickets, anemia, hypercalcemia, end stage renal disease, calcification (e.g., coronary artery calcification and vascular calcification), cardiovascular disease, nephrology, Paget's disease, osteoporosis, hypertension, and renal osteodystrophy. These kits comprise (a) a pharmaceutical composition comprising a compound of Formula I or a salt, hydrate, or solvate thereof, wherein said pharmaceutical composition is in a container; and (b) instructions describing a method of using the pharmaceutical composition to treat primary hyperparathyroidism (e.g., familial hyperparathyroidism), secondary hyperparathyroidism, kidney disease (e.g., chronic kidney disease), hypophosphatemic rickets, anemia, hypercalcemia, end stage renal disease, calcification (e.g., coronary artery calcification and vascular calcification), cardiovascular disease, nephrology, Paget's disease, osteoporosis, hypertension, and renal osteodystrophy.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

### EXAMPLES

### Example 1

Synthesis of Intermediate (R)-1-(naphthalen-1-yl)-1,2,2,2-d4-ethanamine (12a). Intermediate 12a was prepared as outlined in Scheme 6 below. Details of the synthesis are set forth below.

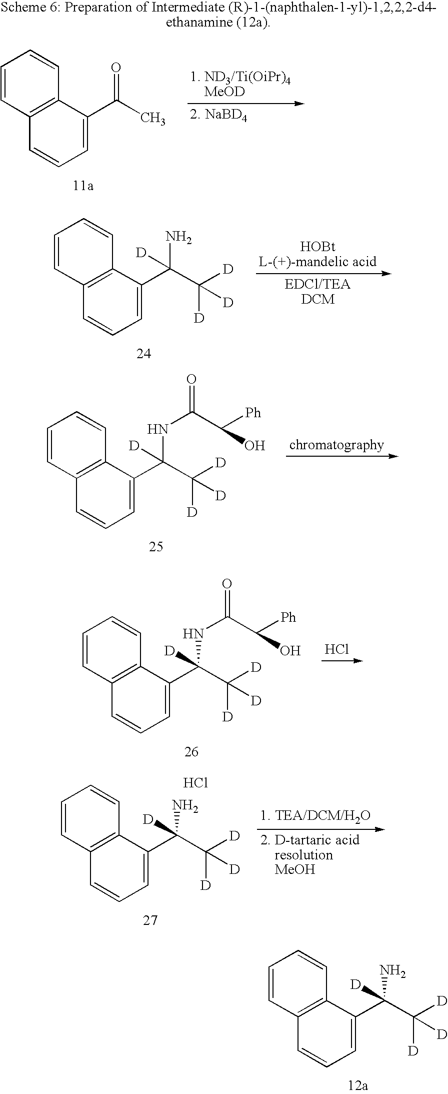

Synthesis of 1-(naphthalen-1-yl)-1,2,2,2-d₄-ethanamine (24). To a flask fit with a mechanical stirrer, reflux condenser, nitrogen inlet and thermowell was added CH₃OD (1 L). Deuterated ammonia was bubbled in, and then 1-acetonaphthone 11a (100 g, 0.588 mol) and titanium isopropoxide (350 mL, 1.18 mol) were added. The resulting mixture was stirred overnight. Sodium borodeuteride (75 g, 1.76 mol) was then added and the reaction was stirred overnight. D₂O (1 L) was then added and the resulting mixture was filtered through a pad of Celite. The filter cake was washed repeatedly liberally with dichloromethane (4×1 L) and the layers were separated. The organic layer was washed with brine, dried over sodium sulfate and the solvent removed under reduced pressure yielding the racemic amine 24 (78 g, 75% yield) as a yellow oil.

Synthesis of (R)-2-hydroxy-N—((R)-1-(naphthalen-1-yl)-1,2,2,2-d₄-ethyl)-2-phenylacetamide (26). To a solution of the racemic amine 24 (78 g, 0.44 mol) in dichloromethane (1 L) stirring at 0° C. was added 1-hydroxybenzotriazole (HOBt) (75 g, 0.55 mol), L-(+)-mandelic acid (80.0 g, 0.484 mol), EDCI (105 g, 0.55 mol), and triethylamine (86 mL, 0.63 mol), all at a rate so that the temperature did not exceed 20° C. After stirring overnight at room temperature, the reaction mixture was diluted with dichloromethane (1 L), and saturated aqueous sodium bicarbonate (2×1 L), and brine (1 L). The organic layer was dried over sodium sulfate and the solvents removed in vacuo to afford the diastereomeric mixture of amines 25 (204 g) as a brown oil (204 g). Purification via chromatography (2 kg silica gel, eluted by heptane with 0-40% ethyl acetate gradient, 100 L) afforded the desired diastereomer 26 (61 g, 45% yield).

Synthesis of (R)-1-(naphthalen-1-yl)-1,2,2,2-d₄-ethanamine hydrochloride (27). A solution of amide 26 (61 g, 0.197 mol) in 6 N hydrochloric acid (0.5 L) was heated at reflux overnight. The mixture was diluted with water (1 L) and cooled on an ice/methanol bath to −5° C. and the pH was adjusted to approximately 9 with 50% aqueous sodium hydroxide solution. The mixture was extracted with ethyl acetate (2×1 L). The organic layer was washed with water (500 mL) and brine (500 mL), dried over sodium sulfate and solvent was removed under reduced pressure. The residual oil was taken up in diethyl ether (500 mL) and HCl/ether was added dropwise with external cooling. The mixture was filtered and the solids dried in a vacuum oven to give the amine HCl salt 27 (32 g, 87% yield) with an enantiomeric ratio of ˜93:7 or 86% ee.

Synthesis of (R)-1-(naphthalen-1-yl)-1,2,2,2-d₄-ethanamine (12a). To a suspension of 27 (21.4 g) in dichloromethane (500 mL)/water (500 mL) was added triethylamine (excess), and the mixture was extracted with dichloromethane. The combined extracts were washed with brine, dried, filtered, and concentrated in vacuo to afford the free amine (19 g) as a brown oil. To a solution of D-tartaric acid (13.29 g) in anhydrous methanol (180 mL) which had previously been heated to 50° C. was added the above free amine (15.8 g). The temperature increased to 65° C., and then reduced, with precipitation beginning immediately when the temperature had fallen to 55° C. The resulting white solid (approximately 23 g) of (R)-12a-(D)-tartrate salt were separated by filtration above 55° C. The solid was redissolved in water (250 mL) at 85-90° C., and the solution was allowed to cool to room temperature slowly and stand at room temperature over the weekend. The resulting white crystalline solid was filtered, washed with a small amount of cold water, then treated with 1 M NaOH solution until all of the solids were dissolved (pH=9). The aqueous mixture was extracted with EtOAc (3×), and the combined organics were dried, filtered, and concentrated in vacuo to afford the desired amine 12a (11.0 g, >99% ee) as a pale-yellow oil.

### Example 2

Synthesis of Intermediate (R)-1-(naphthalen-1-yl)-1-d₁-ethanamine (12b). Intermediate 12b was prepared according to the synthesis outlined in Scheme 7 below. Details of the synthesis are set forth below.

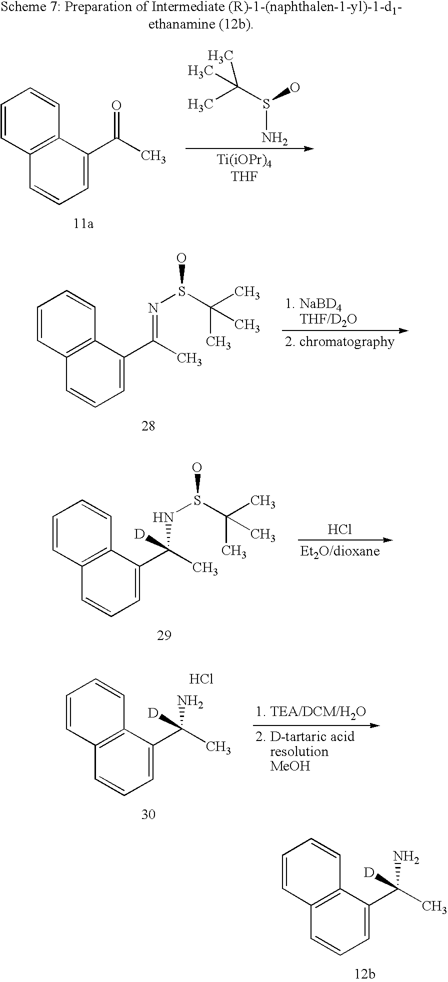

Synthesis of (E)-2-methyl-N-(1-(naphthalen-1-yl)ethylidene)propane-2-sulfinamide (28). To a solution of 1-acetonaphthone 11a (135 g, 0.79 mol) in THF (1.4 L) was added (R)-t-butylsulfinamide (91 g, 0.75 mol), and Ti(OiPr)₄ (450 g, 1.58 mol). The mixture was heated at reflux for 24 hours. The reaction mixture was cooled to room temperature, and then was poured into a rapidly stirring brine solution (1.5 L) and stirred for 15 minutes. The mixture was filtered through a pad of Celite and the filter cake was thoroughly washed with EtOAc (4×1 L). The layers were separated and the organic layer was washed with brine, dried over sodium sulfate and solvent was removed under reduced pressure. The crude product was purified by column chromatography (2 kg silica gel, eluted by heptane to 30% ethyl acetate/heptane gradient) yielding the product 28 as a yellow/orange oil (109 g, 50% yield).

Synthesis of 2-methyl-N—((R)-1-(naphthalen-1-yl)-1-d₁-ethyl)propane-2-sulfinamide (29). To a solution of 28 (109 g, 0.399 mol) in THF (1068 mL) and D₂O (22 mL) that had been cooled to −50° C. was added NaBD₄ (50 g, 1.197 mol). The mixture was warmed slowly to room temperature over a period of 3 hr. The solvent was removed under reduced pressure and the residue was triturated with dichloromethane, filtered and solvent removed under reduced pressure. The crude diastereomers were separated by column chromatography (Analogix silica gel column, eluted by 0-50% ethyl acetate/heptane gradient). The desired diastereomer 29 was isolated as the faster eluting material (47 g, 42% yield).

Synthesis of (R)-1-(naphthalen-1-yl)-1-d₁-ethanamine hydrochloride (30). To a solution of 29 (47 g, 0.17 mol) in dioxane (500 mL) cooled to 0° C. was added HCl/Et₂O (200 mL). The mixture was warmed to room temperature, stirred for 1 hr, diluted with ether and the solids filtered and dried to give the product HCl salt 30 as a white solid (31 g, 88% yield) with an enantiomeric ratio of ˜95:5, or 90% ee.

Synthesis of (R)-1-(naphthalen-1-yl)-1-d₁-ethanamine (12b). To a suspension of 30 (31.0 g) in dichloromethane (500 mL)/water (500 mL) was added triethylamine (excess), and the mixture was extracted with dichloromethane. The combined extracts were washed with brine, dried, filtered, and concentrated in vacuo to afford the free amine (25.8 g) as a brown oil. To a solution of D-tartaric acid (21.7 g) in anhydrous methanol (300 mL) which had previously been heated to 50° C. was added the above free amine (25.8 g). The temperature increased to 65° C. then dropped with crystallization beginning immediately when the temperature had fallen to 55° C. The resulting crystals (approximately 42 g of white solid) of (R)-12b-(D)-tartrate salt were separated by filtration above 55° C. The solid was redissolved in water (450 mL) at 85-90° C., and the solution was allowed to cool to room temperature slowly and to stand at room temperature over the weekend. The resulting white crystalline solid was filtered, washed with a small amount of cold water, then treated with 1 M NaOH solution until all of the solids were dissolved (pH=9). The mixture was extracted with EtOAc (3×), and the combined organics were dried, filtered, and concentrated in vacuo to afford the desired amine 12b (16.9 g, >99% ee) as a pale-yellow oil.

### Example 3

Synthesis of Intermediate 3-(3-(trifluoromethyl)phenyl)-2,2,3,3-d₄-propanoic acid (33). Intermediate 33 was prepared according to Scheme 8 below. Details of the synthesis are set forth below.

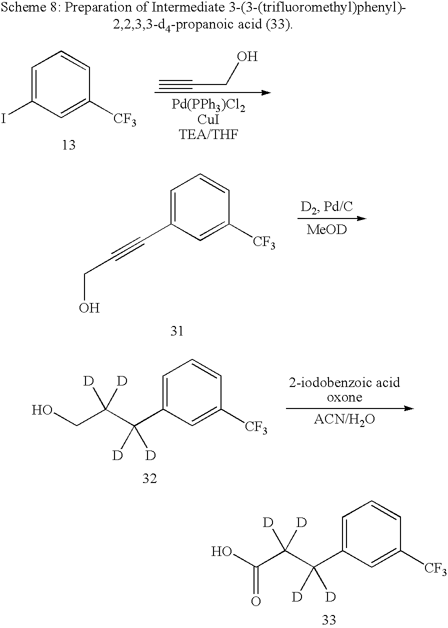

Synthesis of 3-(3-(trifluoromethyl)phenyl)prop-2-yn-1-ol (31). To a solution of 1-trifluoromethyl-3-iodobenzene 13 (20 g, 73.5 mmol) in THF (200 mL) was added Pd(PPh₃)₂Cl₂ (1.3 g, 0.18 mmol) and CuI (0.35 g, 0.18 mmol). Nitrogen was passed through the mixture while stirring for 20 minutes. Propargyl alcohol (4.9 g, 88.2 mmol) was added to the mixture followed by triethylamine (8.9 g, 88.2 mmol). The resulting exothermic reaction raised the temperature of the mixture to approximately 40° C. which was maintained with external heating for 2 hr, then the reaction mixture was stirred overnight. The mixture was cooled to room temperature, diluted with MTBE (200 mL) and filtered through a pad of Celite. The filtrate was washed with brine (200 mL), dried over sodium sulfate and solvent was removed under reduced pressure. The crude product was purified by column chromatography (200 g silica gel, eluted by 10-50% ethyl acetate/heptane gradient) to give the product 31 (14 g, 95% yield) as a yellow oil.

Synthesis of 3-(3-(trifluoromethyl)phenyl)-2,2,3,3-d₄-propan-1-ol (32). To a solution of 31 (12 g, 60 mmol) in CH₃OD (100 mL) in a Parr bottle was added Pd/C (1 g, 10% by weight). The mixture was treated with D₂ (50 psi) for 24 hr. The Pd/C was filtered off and the solvent removed to give 32 (12.5 g, 99% yield) as an oil.

Synthesis of 3-(3-(trifluoromethyl)phenyl)-2,2,3,3-d₄-propanoic acid (33). To a solution of 32 (10.0 g, 48.028 mmol) in acetonitrile (440 mL)/water (220 mL) was added 2-iodobenzoic acid (4.8 g, 19.2112 mmol) and oxone (35.43 g, 57.6336 mmol) at room temperature. The resulting mixture was maintained at 70° C. for 6 hr, cooled in an ice-bath to completely precipitate the insoluble hypervalent iodine by-product, then filtered. The precipitate was washed successively with water (2×300 mL) and dichloromethane (2×300 mL). The combined filtrate was extracted with dichloromethane, and the organic extract was then dried (Na₂SO₄), filtered, and concentrated in vacuo. The residue was subjected to flash chromatography (silica gel with dry-loading, eluted by 30% EtOAc/heptane) to afford the desired compound 33 (8.0 g, 75% yield) as a pale-yellow oil.

### Example 4

Synthesis of Intermediate 3-(3-(trifluoromethyl)phenyl)-3,3-d₂-propanoic acid (38). Intermediate 38 was prepared as outlined in Scheme 9 below. Details of the synthesis are set forth below.

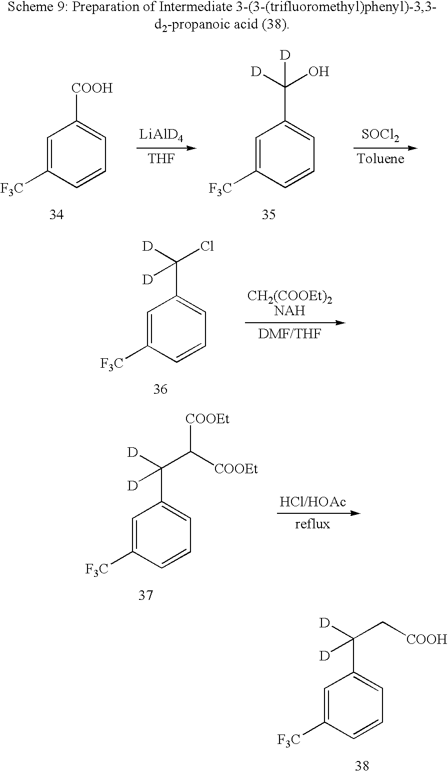

Synthesis of (3-(trifluoromethyl)phenyl)-d₂-methanol (35). To a mixture of LiAlD₄ (16.8 g, 0.40 mol) in THF (1.5 L) was added 3-trifluoromethylbenzoic acid 34 (76 g, 0.40 mol) in portions at 0° C. The resulting mixture was heated at reflux for 8 hr then cooled to room temperature and stirred overnight. The reaction was quenched by the addition of D₂O (16.5 mL), 15% NaOD solution in D₂O (16.5 mL) and D₂O (50 mL) under the cooling of an ice-bath. The solid was filtered, and the filtrate was concentrated in vacuo to give compound 35 (69.4 g, 97% yield) as a pale-yellow oil.

Synthesis of 1-(chloro-d₂-methyl)-3-(trifluoromethyl)benzene (36). To a solution of compound 35 (69.4 g, 0.3877 mol) in toluene (1 L) was added SOCl₂ (31.1 mL, 0.4265 mol) dropwise at room temperature. The resulting mixture was stirred at room temperature for 1 hr, and then was concentrated in vacuo to give crude product 36 (69.0 g) as a pale-yellow oil.

Synthesis of diethyl 2-(3-(trifluoromethyl)phenyl)-d₂-methyl-malonate (37). To a solution of NaH (60% dispersion in mineral oil, 15.87 g, 0.3967 mol) in 1:1 DMF/THF (400 mL) was added a solution of diethyl malonate (150.6 mL, 0.9918 mol) in THF (200 mL) at −10° C. The resulting mixture was stirred at 0° C. for 0.5 hr followed by the dropwise addition of a solution of compound 36 (65.0 g, 0.3306 mol) in THF (150 mL) at −10° C. The resulting mixture was stirred at room temperature overnight, was quenched by water, then extracted with EtOAc. The combined organics were washed with brine, dried (Na₂SO₄), filtered, and concentrated in vacuo. The residue was subjected to flash chromatography (Biotage-150, eluted by 5% EtOAc/heptane) to afford compound 37 (167.0 g) as a colorless oil.

Synthesis of 3-(3-(trifluoromethyl)phenyl)-3,3-d₂-propanoic acid (38). To a mixture of compound 37 (160 g) in acetic acid (1.4 L) was added conc. HCl solution (400 mL) at room temperature, and the resulting mixture was heated at reflux overnight. The mixture was concentrated in vacuo to remove excess acetic acid. The residue was re-dissolved in EtOAc, washed with brine, dried (Na₂SO₄), filtered, then concentrated in vacuo. The residue was subjected to flash chromatography (600 g silica gel, eluted by heptane to 25% EtOAc/heptane in gradient) to afford the desired compound 38 (34.0 g, 40% overall yield for 3 steps) as a pale-yellow oil.

### Example 5

Synthesis of (R)—N-(1-(naphthalen-1-yl)-1,2,2,2-d₄-ethyl)-3-(3-(trifluoromethyl)phenyl)-1-d₂-propan-1-amine hydrochloride (Compound 102). Compound 102 was prepared according to Scheme 10 below. The details of each step in the synthesis are set forth as General Method A.

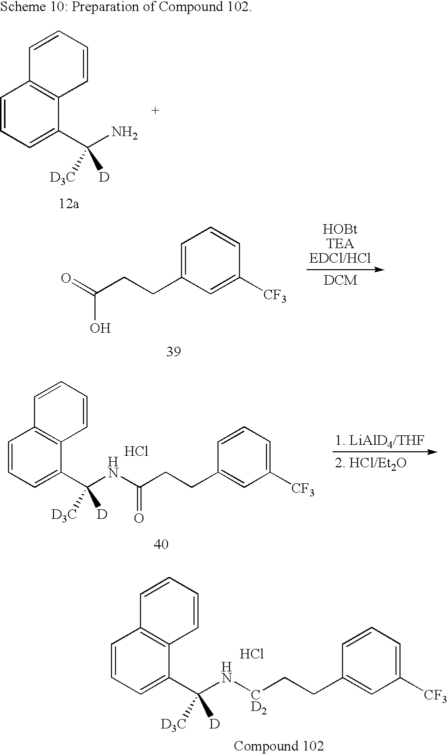

Synthesis of (R)—N-(1-(naphthalen-1-yl)-1,2,2,2-d₄-ethyl)-3-(3-(trifluoromethyl)phenyl)propanamide (40). To a mixture of acid 39 (4.8 g, 22.0 mmol), amine 12a (prepared as in Example 1, 3.5 g, 20.0 mmol), and 1-hydroxybenzotriazole (HOBt) (3.3 g, 24.0 mmol) in dichloromethane (100 mL) was added triethylamine (5.6 mL, 40.0 mmol) and EDCI.HCl (4.6 g, 24.0 mmol) at −50° C. The resulting mixture was allowed to warm to room temperature and stirred overnight. The mixture was then washed sequentially with saturated NaHCO₃ solution, 1M HCl solution, water, then brine, was dried (Na₂SO₄), filtered, then concentrated in vacuo to afford the desired compound 40 (7.8 g) as a white solid. This crude product was used directly in the next step without further purification.

Synthesis of (R)—N-(1-(naphthalen-1-yl)-1,2,2,2-d₄-ethyl)-3-(3-(trifluoromethyl)phenyl)-1-d₂-propan-1-amine hydrochloride (102). To a solution of 40 (5.0 g, 13.318 mmol) in THF (150 mL) was added solid LiAlD₄ (1.12 g, 26.636 mmol) at −50° C. The resulting mixture was allowed to warm to room temperature and stirred overnight, then was heated at reflux for 6 hr. The reaction was quenched by addition of D₂O (1 mL), 15% NaOD solution in D₂O (1 mL), and D₂O (3 mL) under the cooling of an ice-bath. The mixture was filtered, and the filtrate was concentrated in vacuo. The residue was subjected to flash chromatography (Analogix, eluted by hexane to 30% EtOAc/hexane in gradient) to give the free amine of compound 102 as a pale-yellow oil.

To a solution of the free amine (2.8 g, 7.7 mmol) in ether (100 mL) was added 2M HCl/ether solution (7.7 mL, 15.4 mmol) dropwise, and the resulting mixture was stirred at room temperature for 10 min. The suspension was filtered, the solid washed with ether, and dried under vacuum to afford Compound 102 (3.0 g) as a white solid. ¹H-NMR (300 MHz, DMSO-d₆): δ 1.99 (t, J=7.6, 2H), 2.72 (t, J=7.7, 2H), 7.47-7.56 (m, 4H), 7.58-7.65 (m, 3H), 7.96-8.02 (m, 3H), 8.25 (d, J=8.0, 1H), 9.27-9.31 (m, 1H), 9.92-9.96 (m, 1H). ¹³C-NMR (75 MHz, DMSO-d₆): δ 27.52, 32.13, 123.30, 123.45, 123.50, 124.98, 125.36, 125.40, 126.19, 126.83, 127.58, 129.58, 129.97, 130.04, 130.97, 133.08, 134.03, 134.69, 142.92. HPLC (method: 20 mm C18-RP column—gradient method 2-95% ACN in 4 min with 2 min hold at 95% ACN; Wavelength: 254 nm): retention time: 2.98 min. Chiral HPLC (method: Chiral OD column—isocratic mobile phase: 99% hexane+1% ethanol; Wavelength: 220 nm): retention time: 14.06 min., ee>99%. MS (M+H⁺): 364.2. Elemental Analysis (C₂₂H₁₇D₆ClF₃N): Calculated: C=66.07, H=5.80, Cl=8.87, N=3.50, F=14.25. Found: C=65.98, H=5.87, Cl=8.80, N=3.44, F=14.23.

### Example 6

Synthesis of (R)—N-(1-(naphthalen-1-yl)-1,2,2,2-d₄-ethyl)-3-(3-(trifluoromethyl)phenyl)-1,1,3,3-d₄-propan-1-amine hydrochloride (Compound 101). Compound 101 was prepared according to Scheme 10 above, utilizing appropriately deuterated reagents and following General Method A described above.

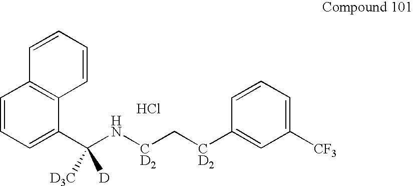

Synthesis of (R)—N-(1-(naphthalen-1-yl)-1,2,2,2-d₄-ethyl)-3-(3-(trifluoromethyl)phenyl)-1,1,3,3-d₄-propan-1-amine hydrochloride (Compound 101). As indicated, compound 101 was prepared via General Method A, above, from acid 38 (prepared as in Example 4, 4.85 g, 22.0 mmol), and amine 12a (prepared as in Example 1, 3.5 g, 20.0 mmol) to afford 5.2 g of pure Compound 101. ¹H-NMR (300 MHz, DMSO-d₆): δ 1.97 (s, 2H), 7.45-7.58 (m, 4H), 7.60-7.65 (m, 3H), 7.97-8.03 (m, 3H), 8.24 (d, J=7.6, 1H), 9.22-9.26 (m, 1H), 9.83-9.87 (m, 1H). ¹³C-NMR (75 MHz, DMSO-d₆): δ 27.42, 123.30, 123.46, 123.52, 124.94, 125.35, 125.40, 126.19, 126.85, 127.58, 129.58, 129.62, 130.04, 130.96, 133.08, 134.04, 134.67, 142.85. HPLC (method: Zorbax 4.6×50 mm SB-Aq 3.5 μm column—gradient method 2-98% ACN+0.1% formic acid in 6 min @ 0.63 mL/min; Wavelength: 268 nm): retention time: 5.44 min. Chiral HPLC (method: Chiral OD column—isocratic mobile phase: 99% hexane+1% ethanol; Wavelength: 220 nm): retention time: 14.07 min., ee>99%. MS (M+H⁺): 366.3. Elemental Analysis (C₂₂H₁₅D₈ClF₃N): Calculated: C=65.74, H=5.77, Cl=8.82, N=3.48, F=14.18. Found: C=65.81, H=5.89, Cl=8.83, N=3.50, F=13.79.

### Example 7

(R)—N-(1-(naphthalen-1-yl)-1,2,2,2-d₄-ethyl)-3-(3-(trifluoromethyl)phenyl)-d₆-propan-1-amine hydrochloride (Compound 100). Compound 100 was prepared according to Scheme 10 above, utilizing appropriately deuterated reagents and following General Method A described above.

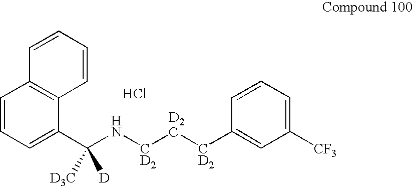

Compound 100 was prepared via General Method A, above, from acid 33 (prepared as in Example 3, 4.89 g, 22.0 mmol), and amine 12a (prepared as in Example 1, 3.5 g, 20.0 mmol) to afford 6.0 g of pure Compound 100. ¹H-NMR (300 MHz, DMSO-d₆): δ 7.45-7.57 (m, 4H), 7.59-7.65 (m, 3H), 7.95-8.03 (m, 3H), 8.24 (d, J=7.6, 1H), 9.19 (d, J=11.7, 1H), 9.76 (d, J=11.7, 1H). ¹³C-NMR (75 MHz, DMSO-d₆): δ 123.30, 123.46, 123.52, 124.89, 125.35, 125.40, 126.19, 126.86, 127.59, 129.59, 129.64, 130.05, 130.96, 133.09, 134.04, 134.67, 142.83. HPLC (method: Zorbax 4.6×50 mm SB-Aq 3.5 μm column—gradient method 2-98% ACN+0.1% formic acid in 6 min @ 0.63 mL/min; Wavelength: 268 nm): retention time: 5.45 min. Chiral HPLC (method: Chiral OD column—isocratic mobile phase: 99% hexane+1% ethanol; Wavelength: 220 nm): retention time: 14.46 min., ee>99%. MS (M+H⁺): 368.1. Elemental Analysis (C₂₂H₁₃D₁₀ClF₃N): Calculated: C=65.42, H=5.74, Cl=8.78, N=3.47, F=14.11. Found:

C=65.58, H=6.12, Cl=8.73, N=3.59, F=13.60.

### Example 8

(R)—N-(1-(naphthalen-1-yl)-1-d₁-ethyl)-3-(3-(trifluoromethyl)phenyl)-1,1-d₂-propan-1-amine hydrochloride (Compound 105). Compound 105 was prepared according to Scheme 10 above, utilizing appropriately deuterated reagents and following General Method A described above.

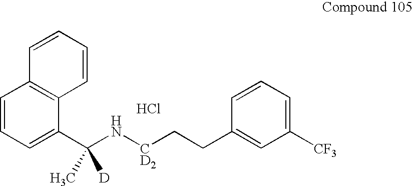

Synthesis of (R)—N-(1-(naphthalen-1-yl)-1-d₁-ethyl)-3-(3-(trifluoromethyl)phenyl)-1,1-d₂-propan-1-amine hydrochloride (Compound 105). As indicated, compound 105 was prepared via General Method A, above, from acid 39 (4.80 g, 22.0 mmol), and amine 12b (prepared as in Example 2, 3.45 g, 20.0 mmol) to afford 5.3 g of pure Compound 105. ¹H-NMR (300 MHz, DMSO-d₆): δ 1.67 (s, 3H), 1.98 (t, J=7.7, 2H), 2.72 (t, J=7.8, 2H), 7.47-7.55 (m, 4H), 7.59-7.65 (m, 3H), 7.94-8.02 (m, 3H), 8.23 (d, J=7.3, 1H), 9.12-9.25 (m, 1H), 9.67-9.88 (m, 1H). ¹³C-NMR (75 MHz, DMSO-d₆): δ 20.36, 27.60, 32.13, 123.32, 123.55, 124.86, 125.38, 125.43, 126.22, 126.90, 127.62, 129.62, 129.68, 130.09, 130.97, 133.12, 134.07, 134.72, 142.92. HPLC (method: 20 mm C18-RP column—gradient method 2-95% ACN+0.1% formic acid in 3.3 min with 1.7 min hold at 95% ACN; Wavelength: 254 nm): retention time: 2.97 min. Chiral HPLC (method: Chiral OD column—isocratic mobile phase: 99% hexane+1% ethanol; Wavelength: 220 nm): retention time: 13.92 min., ee>99%. MS (M+H⁺): 361.2. Elemental Analysis (C₂₂H₂₀D₃ClF₃N): Calculated: C=66.58, H=5.84, Cl=8.93, N=3.53, F=14.36. Found: C=66.59, H=5.85, Cl=8.92, N=3.50, F=14.22.

### Example 9

(R)—N-(1-(naphthalen-1-yl)-1-d₁-ethyl)-3-(3-(trifluoromethyl)phenyl)-1, 133-d₄-propan-1-amine hydrochloride (Compound 104). Compound 104 was prepared according to Scheme 10 above, utilizing appropriately deuterated reagents and following General Method A described above.

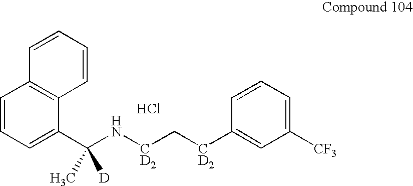

Synthesis of (R)—N-(1-(naphthalen-1-yl)-1-d₁-ethyl)-3-(3-(trifluoromethyl)phenyl)-1,1,3,3-d₄-propan-1-amine hydrochloride (Compound 104). As indicated, compound 104 was prepared via General Method A, above, from acid 38 (prepared as in Example 4, 4.84 g, 22.0 mmol), and amine 12b (prepared as in Example 2, 3.45 g, 20.0 mmol) to afford 3.9 g of pure Compound 104. ¹H-NMR (300 MHz, DMSO-d₆): δ 1.68 (s, 3H), 1.97 (s, 2H), 7.45-7.55 (m, 4H), 7.57-7.65 (m, 3H), 7.97-8.03 (m, 3H), 8.24 (d, J=7.5, 1H), 9.22-9.27 (m, 1H), 9.83-9.88 (m, 1H). ¹³C-NMR (75 MHz, DMSO-d₆): δ 20.41, 27.44, 123.31, 123.49, 124.94, 125.38, 125.43, 126.22, 126.86, 127.60, 129.60, 129.64, 130.06, 130.97, 133.11, 134.05, 134.74, 142.87. HPLC (method: 20 mm C18-RP column—gradient method 2-95% ACN+0.1% formic acid in 3.3 min with 1.7 min hold at 95% ACN; Wavelength: 254 nm): retention time: 2.99 min. Chiral HPLC (method: Chiral OD column—isocratic mobile phase: 99% hexane+1% ethanol; Wavelength: 220 nm): retention time: 14.02 min., ee>99%. MS (M+H⁺): 363.3. Elemental Analysis (C₂₂H₁₈D₅ClF₃N): Calculated: C=66.24, H=5.81, Cl=8.89, N=3.51, F=14.29. Found: C=66.25, H=5.86, Cl=8.91, N=3.48, F=14.28.

### Example 10

(R)—N-(1-(naphthalen-1-yl)-1-d₁-ethyl)-3-(3-(trifluoromethyl)phenyl)-d₆-propan-1-amine hydrochloride (Compound 103). Compound 103 was prepared according to Scheme 10 above, utilizing appropriately deuterated reagents and following General Method A described above.

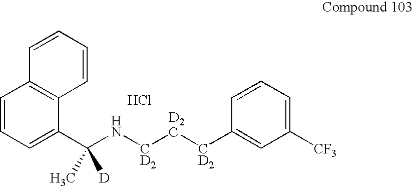

Synthesis of (R)—N-(1-(naphthalen-1-yl)-1-d₁-ethyl)-3-(3-(trifluoromethyl)phenyl)-d₆-propan-1-amine hydrochloride (Compound 103). As indicated, compound 103 was prepared via General Method A, above, from acid 33 (prepared as in Example 3, 4.88 g, 22.0 mmol), and amine 12b (prepared as in Example 2, 3.45 g, 20.0 mmol) to afford 3.2 g of pure Compound 103. ¹H-NMR (300 MHz, DMSO-d₆): δ 1.67 (s, 3H), 7.45-7.55 (m, 4H), 7.57-7.65 (m, 3H), 7.96-8.03 (m, 3H), 8.24 (d, J=7.5, 1H), 9.22 (bs, 1H), 9.77 (bs, 1H). ¹³C-NMR (75 MHz, DMSO-d₆): δ 20.40, 123.32, 123.49, 123.53, 124.89, 125.38, 125.43, 126.22, 126.88, 127.61, 129.62, 129.65, 130.06, 130.98, 133.11, 134.06, 134.76, 142.86. HPLC (method: 20 mm C18-RP column—gradient method 2-95% ACN+0.1% formic acid in 3.3 min with 1.7 min hold at 95% ACN; Wavelength: 254 nm): retention time: 2.96 min. Chiral HPLC (method: Chiral OD column—isocratic mobile phase: 99% hexane+1% ethanol; Wavelength: 220 nm): retention time: 14.05 min., ee>99%. MS (M+H⁺): 365.1. Elemental Analysis (C₂₂H₁₆D₇ClF₃N): Calculated: C=65.91, H=5.78, Cl=8.84, N=3.49, F=14.22. Found: C=66.82, H=5.85, Cl=8.86, N=3.47, F=14.09.

### Evaluation of Compound Stability

Certain in vitro liver metabolism studies have been described previously in the following references, each of which is incorporated herein in their entirety: Obach R S, Drug Metab. Disp. 1999, 27: 1350; Houston, J B et al., Drug Metab. Rev. 1997, 29: 891; Houston J B, Biochem Pharmacol 1994, 47: 1469; Iwatsubo T et al., Pharmacol. Ther. 1997, 73: 147; and Lave T et al., Pharm. Res. 1997, 14: 152.

Materials and Methods: Human liver microsomes (20 mg/mL, pool of 50 individuals) were obtained from Xenotech LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich (St. Louis, Mo.). Test compounds were obtained from Concert Pharmaceuticals.

Determination of Metabolic Stability: 10 mM stock solutions of test compounds were prepared in DMSO. The 10 mM stock solutions were diluted to 1 mM in acetonitrile (ACN). The 20 mg/mL liver microsomes were diluted to 0.625 mg/mL in 0.1 M potassium phosphate buffer, pH 7.4, containing 3 mM MgCl₂. 1 mM test compound was added to the diluted microsomes to obtain a mixture containing 1.25 μM test compound. The microsome-test compound mixtures were added to wells of a 2 mL 96-well deep well polypropylene plate in triplicate. The plate was warmed to 37° C. before initiating the reactions by addition of prewarmed NADPH in 0.1 M potassium phosphate buffer, pH 7.4, containing 3 mM MgCl₂. The final reaction volume was 0.5 mL and contained 0.5 mg/mL microsomes, 1 μM test compound, 2 mM NADPH in 0.1 M potassium phosphate buffer, pH 7.4, and 3 mM MgCl₂. The reaction mixtures were incubated at 37° C. and 50 μL aliquots were removed at 0, 10, 20, and 30 minutes and added to shallow-well 96-well plates which contained 50 μL of ice-cold ACN with internal standard to stop the reactions. The plates were stored at −20° C. for 30 minutes after which 100 μL of water was added to the wells of the plate before centrifugation to pellet precipitated proteins. Supernatants were transferred to another 96-well plate and analyzed for amounts of parent remaining by LC-MS/MS using an Applied Biosystems API 4000 mass spectrometer.

Data analysis: The in vitro t_{1/2}S for test compounds were calculated from the slopes of the linear regression of % parent remaining (ln) vs incubation time relationship.

in vitro t_{1/2}=0.693/k

k=−[slope of linear regression of % parent remaining (ln) vs incubation time]

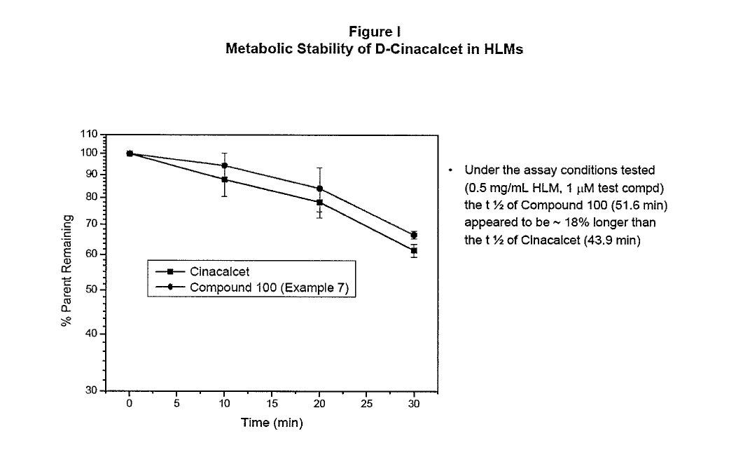

Data analysis was performed using Microsoft Excel Software, and the results for Example 7 (compound 100) are depicted in FIG. 1.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications.

We claim:

1. A compound of Formula I, or a salt, hydrate, or solvate thereof: 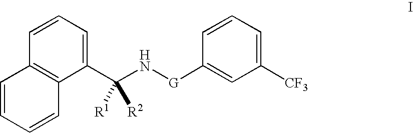
wherein:
R¹ is selected from CH₃, CH₂D, CHD₂, and CD₃;
R² is selected from H and D;
G is †-(CHₘD₂₋ₘ)(CHₙD₂₋ₙ)(CHₚD₂₋ₚ)-, wherein m, n, and p are each independently selected from 0, 1, and 2, and the † represents the portion of G attached to the NH moiety in the compound; and
wherein said compound comprises at least one deuterium atom at R¹, R², or G.

2. The compound according to claim 1, wherein R¹ is CD₃ or CH₃.

3. The compound according to claim 2, wherein R¹ is CD₃.

4. The compound according to any one of claims 1 to 3, wherein R² is D.

5. The compound according to any one of claims 1 to 3, wherein each of m, n, and p is independently selected from 0 or 2.

6. The compound according to claim 5, wherein each of m, n, and p is 2.

7. The compound according to claim 5, wherein n is 0; and each of m and p is 2.

8. The compound according to claim 5, wherein each of n and p are 0; and m is 2.

9. The compound according to claim 5, wherein m is 2; and each of n and p is independently selected from 0 and 2.

10. The compound according to claim 9, wherein R¹ is CH₃.

11. The compound according to claim 1, selected from the group consisting of the following compounds:

| Compound | R¹ | R² | G |
|---|---|---|---|
| 100 | CD₃ | D | †CD₂CD₂CD₂ |
| 101 | CD₃ | D | †CD₂CH₂CD₂ |
| 102 | CD₃ | D | †CD₂CH₂CH₂ |
| 103 | CH₃ | D | †CD₂CD₂CD₂ |
| 104 | CH₃ | D | †CD₂CH₂CD₂ |
| 105 | CH₃ | D | †CD₂CH₂CH₂ |
| 106 | CH₃ | H | †CD₂CH₂CH₂ |

12. The compound according to any one of claims 1 to 3, wherein the salt is a hydrochloride salt.

13. A pyrogen-free composition comprising a compound according to any one of claims 1 to 3 and an acceptable carrier.

14. The composition according to claim 13, wherein the composition is formulated for pharmaceutical use and the carrier is a pharmaceutically acceptable carrier.

15. The composition according to claim 14, wherein the composition is formulated for oral use.

16. The composition according to claim 14, additionally comprising a second therapeutic agent.

17. The composition according to claim 16, wherein the second therapeutic agent is selected from vitamin D, a vitamin D analogue, a phosphate binder, and an agent used to raise serum calcium concentrations.

18. The composition according to claim 14, wherein the composition is used for the treatment or prevention of a disease or condition selected from primary hyperparathyroidism, secondary hyperparathyroidism, kidney disease, hypophosphatemic rickets, anemia, hypercalcemia, end stage renal disease, calcification, cardiovascular disease, nephrology, Paget's disease, osteoporosis, hypertension, and renal osteodystrophy.

19. A method of treating a subject suffering from, or susceptible to, a disease or condition that is beneficially treated by an agent that increases the sensitivity of a calcium receptor on a parathyroid gland, comprising the step of administering to the subject in need thereof a composition according to claim 14.

20. The method according to claim 19, wherein the disease or condition is selected from primary hyperparathyroidism, secondary hyperparathyroidism, kidney disease, hypophosphatemic rickets, anemia, hypercalcemia, end stage renal disease, calcification, cardiovascular disease, nephrology, Paget's disease, osteoporosis, hypertension, and renal osteodystrophy.

21. The method according to claim 20, wherein the disease or condition is selected from secondary hyperparathyroidism and hypercalcemia.

22. The method according to claim 19, comprising an additional step of co-administering to the subject a second therapeutic agent.

23. The method according to claim 22, wherein the second therapeutic agent is selected from vitamin D, a vitamin D analogue, a phosphate binder, and an agent used to raise serum calcium concentrations.

24. The method according to claim 19, wherein the subject is a human.
